(19) United States
(12) Plant Patent Application Publication
(10) Pub. No.: US 2022/0151121 P1
(43) Pub. Date: May 12, 2022

(54) Strawberry plant named 'SGS73 1'

(50) ;

(71) Applicant: INSTITUTO NACIONAL DE INVESTIGACION AGROPECUARIA (INIA), Montevideo (UY)

(72) Inventors: Esteban Vicente, Salto (UY); Ariel Manzzioni, Salto (UY); Joanna Lado, Salto (UY); Matias González, Salto (UY); Ana Arruabarrena, Salto (UY); Mario Gambiasi, Salto (UY)

(21) Appl. No.: 17/300,752

(22) Filed: Oct. 19, 2021

Related U.S. Application Data
(60) Provisional application No. 63/204,816, filed on Oct. 27, 2020.

(51) Int. Cl. A01H 6/74

(52) U.S. Cl. PLT/209

(57) ABSTRACT

A new short-day Strawberry Plant named ‘SGS73 1’ is disclosed, with exceptional fruit shape and flavor characteristics.

### CROSS-REFERENCE TO RELATED APPLICATIONS

This application claims priority of co-pending provisional application Ser. No. 63/204,816 as filed on Oct. 27, 2020.

### STATEMENT REGARDING FEDERALLY SPONSORED RESEARCH OR DEVELOPMENT

None

Genus and species: Fragaria X ananassa Duch.

Variety denomination: ‘SGS73 1’.

### BACKGROUND OF THE INVENTION

‘SGS73 1’ was obtained from the INSTITUTO NACIONAL DE INVESTIGACION AGROPECUARIA (INIA) a breeding program located in Salto, Uruguay. It originated from the cross between two advanced clones carried out in 2013 at the INIA Salto Grande facility in Salto, Uruguay. It was selected in the field of individuals in 2014 and later evaluated in preliminary and advanced agronomic trials against reference varieties, always under conditions representative of the production technology of the Salto area, Uruguay. Simultaneously it was characterized by its sanitary behavior against the natural infection of root/crown, foliar and fruit pathogens (2015-2019). During 2018, it was evaluated at the productive level in the producer's farm, with a panel made up of producers, technicians, nurserymen, and INIA staff. The following year, it was validated on a larger scale at four representative commercial farms. During the last three years their physical, chemical, and postharvest life quality of fruit has been characterized.

Contrast is made with maternal parent ‘SGN48.3’ (INIA Ágata™), in which ‘SGS73 1’ is more tolerant to stem and root diseases with superior fruit quality and agronomic behavior. According to the information available, ‘SGS73 1’ would be recommended for autumn, winter, and spring production in protected cultivation in the northwest region of Uruguay. The new cultivar ‘SGS73 1’ has a superior performance than ‘SGN48.3’ in semi-early productivity (harvests in August and September); vegetative development by habit and ease of harvest; fruit quality, especially in shape and flavor; and health, due to its lower incidence of botrytis, spider mites and powdery mildew in fruit.

Paternal parent is ‘SGP20.2’, which belongs to the INIA Uruguay breeding program and has not yet been commercialized.

### BRIEF DESCRIPTION OF THE PHOTOGRAPHS

FIG. 1 illustrates the fruit of ‘SGS73 1’ harvested August 2019 from a 5-month-old plant;

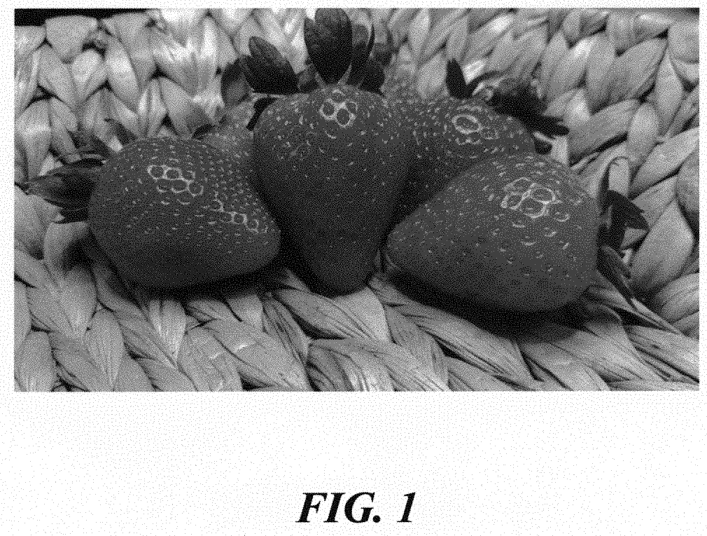

FIG. 2 illustrates plants of ‘SGS73 1’ at 6 months of age cultivated with black plastic mulch and drip irrigation;

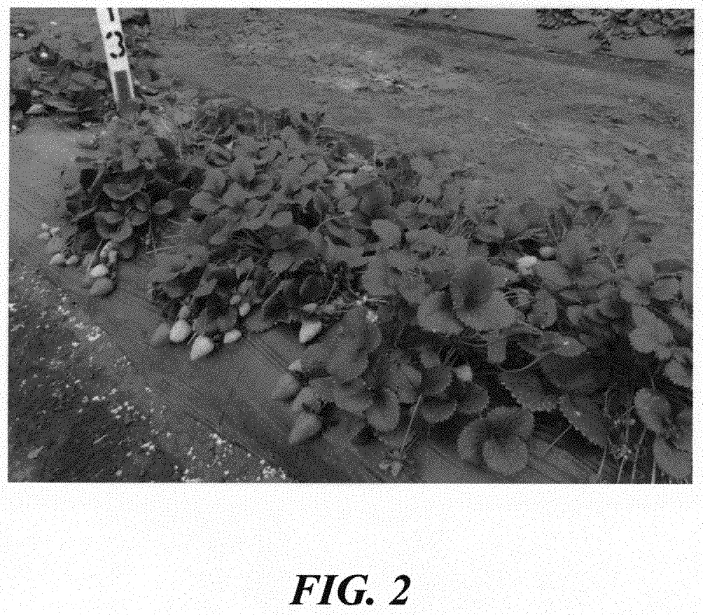

FIG. 3 illustrates plants of ‘SGS73 1’ at 5 months of age cultivated with black plastic mulch and drip irrigation; and

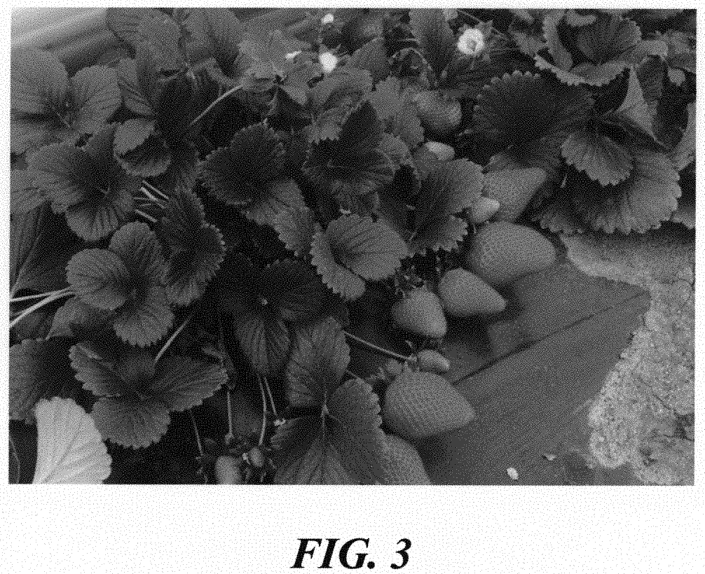

FIG. 4 illustrates greenhouse ‘SGS73 1’ 4-month-old mother plants after transplantation and planted in pots with 20 days of rooting.

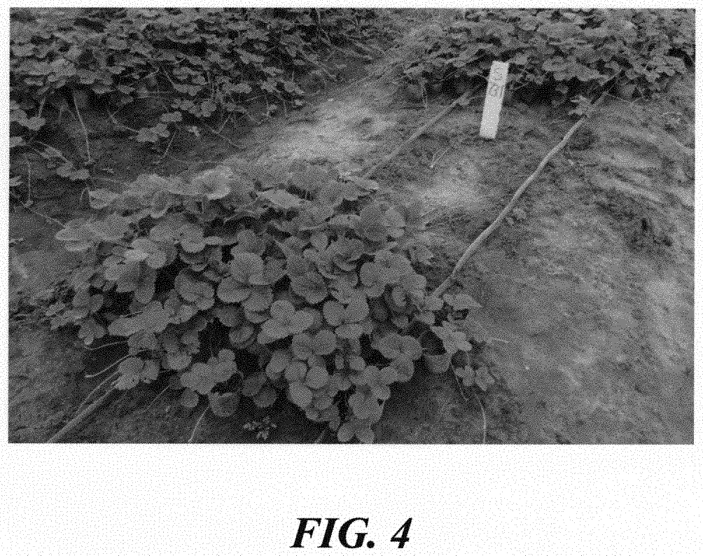

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2019 growing seasons in Salto Grande, Salto, Uruguay. It should be understood that the characteristics described will vary somewhat depending upon cultural practices and climatic conditions and can vary with location and season. Quantified measurements are expressed as an average of measurements taken from a number of individual plants of the new variety. The measurements of any individual plant or any group of plants, of the new variety may vary from the stated average. Color terminology follows CIE 1976 L*a*b* color space (International Commission on Illumination (CIE)).
General description:
   Ploidy.—Octoploid.
   Blooming period.—April thru November in the Southern Hemisphere.
   Plant type.—Not remontant, short day, early fruiting; short-day types as it produces fruit earlier in the season.
   Plant habit.—Erect.
   Root description.—Moderately branching; medium-high density; Medium-high in thickness, fibrous; typically, cream white to white in color.
   Propagation.—By runners, cuttings, and direct rooting in pots.
   Growth rate.—Medium-high vigor.
   Stolon description.—Number of stolons, few; anthocyanin coloration, weak; density of pubescence, medium.
Foliage description:
   Leaf division.—Compound with typically three leaflets per leaf
   Leaf blistering.—Medium.
   Leaf glossiness.—Medium.
   Leaf variegation.—Absent.
   Leaf arrangement.—Typical spiral arrangement.
   Leaflet shape in cross section.—Concave.
   Leaflet length in relation to width.—Equal.
   Leaflet margins.—Crenate.
   Leaflet base.—Obtuse to rounded.
   Leaflet apex.—Rounded.
   Leaflet venation.—Pinnate.
   Leaflet color.—Medium green.
   Petiole.—Length medium: 10 cm to 15 cm; Diameter: about 3 mm to 4.5 mm; attitude of hairs: slightly outwards; density of pubescence: sparse.
   Peliolules.—Length 0.5 cm to 0.8 cm.
   Stipule.—Anthocyanin coloration medium.
Flower description:
   Position of inflorescence in relation to foliage.—Same level.
   Flower initiation and expression conditions.—Very low to no cold requirements to flower; very early variety with a short day.
   Time of flowering (50% of plants at first flower).—Very early; in Uruguay they bloom from April to November.
   Size of calyx in relation to corolla.—Larger.
   Position of calyx attachment.—Inserted.
   Diameter of calyx.—1 cm to 2 cm.
   Petals.—Length in relation to width: moderately longer; color of upper side: white; length: 8 mm to 12 min; width: 8 mm to 10 mm.
   Peduncle.—8 cm to 12 cm.
   Pistils.—Quantity per flower: about 28 to 32.
   Stigma.—Shape rounded.
   Stamens.—Quantity per flower: about 20 to 30.
Fruit description:
   Shape.—Conical-cordiform.
   Season of harvest.—July-September.
   Time of ripening (50% of plants with first ripe fruit).—Very early; in Uruguay the harvest begins in Mid-May.
   Time of bearing.—20 to 25 days depending on weather condition and season of the year.
   Size.—Large to very large.
   Evenness of Surface.—Even or very slightly uneven.
   Glossiness.—High gloss, outstanding fruit appearance.
   External color (skin).—.

External Color

| L* | a* | b* | Hue | Chroma |
|---|---|---|---|---|
| 32.8-38.7 | 32.0-35.2 | 21.5-25.8 | 32.4-36.2 | 39.7-43.7 |

Internal color.—.

Internal Color

| L* | a* | b* | Hue | Chroma |
|---|---|---|---|---|
| 58.1-62.3 | 16.3-19.4 | 24.5-27.6 | 53.2-57.8 | 30.3-33.2 |

Evenness of color of skin.—Even distribution.
Acidity.—0.40-0.55 Titratable acidity (% citric acid).
Sweetness.—5.50-8.80 brix.
Firmness.—Very firm.
Fruit weight.—16 g-25 g.
Shelf life.—Very good postharvest life.
Achene.—Level with surface. Width of band without achenes; absent or very narrow.
Disease and pest resistance:
   Global response to the disease complex root and stem.—High-medium field tolerance.
   Resistance to powdery mildew at leaf level.—Very high.
   Resistance to powdery mildew at fruit level.—High.
   Resistance to botrytis.—Low incidence.
   Resistance to spider mites.—Low preference.
   Resistance to anthracnose in fruit.—Intermediate.

1. A new and distinct strawberry plant as shown and described herein.
